(19) United States
(12) Patent Application Publication
(10) Pub. No.: US 2020/0128797 A1
(43) Pub. Date: Apr. 30, 2020

(54) Three Methods for High-Volume Asexual Propagation of Octocorallia (Alcyonaria) and Corallimorpharia Soft Corals

(71) Applicant: Noel Thomas Curry, Atlanta, GA (US)

(72) Inventor: Noel Thomas Curry, Atlanta, GA (US)

(21) Appl. No.: 16/174,280

(22) Filed: Oct. 30, 2018

(51) Int. Cl. A01K 61/30; A01K 63/04; A01K 63/00; A01G 33/00; A01K 61/80; A01K 67/033

(52) U.S. Cl. CPC A01K 61/30; A01K 63/045; A01K 67/033; A01G 33/00; A01K 61/80; A01K 63/006

(57) ABSTRACT

Oceans are warming becoming more acidic, and coral reefs are rapidly declining primarily due to anthropogenic global warming (climate change). Methods must be found to preserve as many of the 3000 species of soft corals as possible before they are gone. Maintaining biodiversity on coral reefs is essential to a balanced healthy reef. Since the chances of a political solution to reducing greenhouses gases are very unlikely, steps must be taken now to collect, preserve, and propagate as many marine reef organisms as possible including soft corals. Propagation methods of stony corals are well known, but soft corals are far more difficult to propagate due to their lack of a stony skeleton. This invention presents three methods for high volume, efficient, and inexpensive propagation of soft corals Alcyonacea (Octocorallia) and Corallimorpharia. Two methods use cubes in an eggcrate matrix, and the third involves propagating soft corals on glass plates.

### BACKGROUND OF THE INVENTION

The biodiversity of our Earth's coral reefs which took millions of years to build, may be destroyed in the next few decades unless we take swift and serious action to protect the reefs now. Coral reefs are the most diverse ecosystems in the world, and are often call the rainforests of the sea due to the incredible variety and numbers of organisms living on them.

A healthy reef system is a delicate balance of all of the organisms living on it and in it. It's an incredibly complex ecosystem that forms an intricate food and shelter web. Removing even a few species can have a domino effect on the remaining inhabitants that is impossible to predict.

Coral reefs around the world are being decimated by environmental changes due to primarily to anthropogenic climate change (global warming).

Millions of species of organisms exist on our reef systems, with most still unknown to taxonomists. Without protection from and mitigation of the effects of global warming, coral reefs will likely go from the most biodiverse areas in the world, to minimally biodiverse areas supporting only a few species of hardy stony corals, soft corals, fish, and invertebrates. It's the difference between a rainforest supporting millions of species, and a stand of replanted pine trees supporting a few hundred species, if that.

Reefs occupy less than one percent of the ocean floor, and yet are home to more than twenty five percent of all marine life. Loss of biodiversity in coral reefs is a critical issue. Already we have lost untold thousands of species of fish and invertebrates.

There are over 3000 species of soft corals (Octocorallians) alone, They are integral members of the reef ecosystem. Unlike stony hard corals (Hexacorallians) which are reef builders (hermatypic), most soft corals are not reef builders (ahermatypic). But they are still an essential component for the biodiversity of coral reefs. Nearly all soft corals use symbiotic photosynthesizing zooxanthella as a major energy source, so they are dependent upon the sun for survival. They provide important habitats for many species of fish, invertebrates, algae and many other marine species.

Saving the coral reefs doesn't just mean saving the prettiest, most dominant or easily grown species. It means saving as many different species of all reef organisms as possible. Loss of biodiversity is the real potential tragedy for corals, and will be devastating to the health of the reefs if it continues to occur.

Ocean reefs aren't important only to ocean dwellers. Today, billions of people depend on coral reefs for food, fresh water, income, recreation, medicine, tourism, and coastal protection. Coral reefs are home to the fishes and invertebrates that feed 25% of the world's population.

Unfortunately over the past thirty years, pollution, severe over fishing, and now increasingly global warming/climate change have destroyed about half of the shallow-water reefs on the planet. Increasing water temperatures have been causing coral bleaching events that are unprecedented in scale. Many of the most severe coral bleaching events in recorded history have happened in the past three years.

When it's gone, the biodiversity of the world's oceans will not be easily replaced. Natural reestablishment of the reefs will not provide the same level of biodiversity as the wild reefs have now, for millions of years.

That's why it's critical for us to collect, preserve, and propagate as many reef organisms as possible now, before they are gone.

Corals can survive only within a very narrow band of environmental conditions. As the amount of CO2 in the atmosphere has increased from around 270 ppm at the beginning of the industrial age, to more than 400 ppm today, average water temperatures have increased and the oceans are becoming increasingly acidic.

Already we are seeing the more sensitive corals dying off en masse. It is estimated that the world's largest coral reef system, the Great Barrier Reef, has lost half of it's corals since 2016. Most likely we will see a gradual die off of more susceptible corals over time, leaving only the species that can tolerate warmer temperatures and increased acidity. But even those may not survive long term if conditions continue to deteriorate.

Many reputable scientists predict that with only a 1.5 degree C. increase in the Earth's temperature, we will lose 90% of the world's corals. They further predict that with a 2 degree C. increase in our temperature, all of the corals on Earth will be gone. We already are at a nearly 1 degree C. increase according to NASA in a 2018 report.

Because corals are sessile organisms, they have evolved very powerful chemicals to fight off their neighbors. Defending their turf for light, nutrients, and water are essential if they are to survive in nature. Those same chemicals now are being investigated for medical research uses, such as fighting cancer, infections, and other important uses. The ocean is potentially a massive bio-medical resource for drugs and it is largely untapped so far.

It's essential that we do as much as possible now to not only to preserve and protect the existing reefs we still have, but also to collect specimens of all of the remaining corals for preservation, propagation, and replanting in more environmentally friendly areas until we can solve the climate change problem permanently. It would be a tragedy to solve the climate change problem eventually, but still lose the reefs because we waited too long.

It's imperative to discover a very efficient system of coral propagation that can produce very large quantities of healthy juvenile soft corals 119 for the least amount of labor and cost. Currently we have the ability to produce fairly large numbers of stony corals, but the propagation of large numbers of soft corals has been elusive, due to a major difference in their structure, until now.

Propagation by fragmentation of stony hermatypic hexacoralians is quite simple. Widely known methods exist, using coral fragments from mother colonies. Since Small Polyped Stony (SPS) corals (aka hard corals or Scleractinians) have a rigid calcium carbonate skeleton, fragments can be removed from the mother colony and attached to a solid substrate using Cyanoacrylate (crazy glue) or a waterproof plastic epoxy putty that hardens quickly when attached to a substrate.

However soft corals are very difficult to reproduce asexually since there they have no stony skeleton to attach to a substrate. Instead of a solid stony skeleton, soft corals have very small spiny calcium carbonate skeletal structures in their tissue called sclerites which give them some degree of support when the coral is inflated with sea water, but don't aid in the attachment to the substrate.

Recent studies have shown that in waters where the pH is lower, corals and other invertebrates such as oysters and clams, are unable to control the calcification process which affects their ability to build their calcium carbonate skeletons. Even though soft corals don't have a full calcium carbonate skeleton like stony corals do, their sclerites are made of calcium carbonate, so their ability to produce these structures is likely impaired in more acidic waters.

Growing-out soft corals already attached to a substrate is not the problem. That's quite easy if you provide the correct environmental conditions.

The problem is getting the soft coral cutting to attach to a new substrate. Initial soft coral cutting 108 attachment to the substrate is the big challenge that this invention solves.

It's often said that reproducing soft corals asexually by fragmentation is “like getting jello to attach to a rock”. That is one reason why, prior to my invention, there was no known way to reproduce large quantities of soft corals. Fortunately I was able to discover several efficient methods that allow the attachment of very large quantities of asexually produced soft coral cuttings 108 to a substrate.

Having the ability to produce large numbers of soft corals efficiently will enable us to reestablish damaged reefs, and collect, propagate and preserve species from reefs that are still currently healthy. Together with already established methods for propagating stony corals, we have a chance of reestablishing and maintaining some level of biodiversity on the coral reefs before it's too late.

It's quite possible that the only corals that exist in the future will be in captivity.

### BRIEF SUMMARY OF THE INVENTION

In view of the disadvantages of the currently known methods of asexual soft coral propagation, the present invention provides several new systems for asexually propagating large numbers of soft corals encompassing a large number of soft coral species.

### Advantages of this Invention

High volume propagation of many different types of soft corals previously produced in small quantities.

Allows the propagation of many species of soft corals that were not previously possible at all.

In addition, these methods of asexual propagation are:
Less time consuming to produce.
A highly efficient use of space during initial soft coral attachment phase.
Less labor intensive.
Less costly to produce.
More efficient use of space during grow-out period.
Easier to ship, saving weight, space, and money, benefiting small coral farms around the world that have to carefully watch their funds.

And very importantly they have a far lower mortality rate of the soft coral cuttings 108.

Method A—High Volume Asexual Propagation Technique for Alcyonacean Soft Corals Using the DIMPLE Cube Method (DCM)

The object of this invention was to create a method to attach individual soft coral cuttings 108 onto the top of a small porous cementitious cubed substrate. Each cube had a small indentation (dimple) on top, to hold the soft coral cutting 108 so it would not be pressing against the upper retaining net 110 as it became attached over the following weeks. Each dimple contained one or more asexually produced soft coral cutting 108 (of the same or different species). The dimple cubes 102 were arrayed in a polystyrene eggcrate matrix approximately 22 cm×22 cm, with 117 cubes total, referred to as the dimple cube plate (DCP) 104. The number of cubes per plate could vary according to your system design.

Soft coral cuttings 108 were prepared and placed into each dimple 103. A net 110 was placed over the plate and rubber bands attached on the outside (FIG. 21), completing the Dimple Cube Plate Assembly (DCPA) (FIGS. 5, 6).

After the soft coral cuttings 108 became naturally attached to the substrate, the DCPA was disassembled and checked for adhesion of the cuttings. The DCP with newly attached corals could then be efficiently moved, shipped, or stored in a saltwater system for grow-out. The soft coral cuttings 108 could then be allowed to grow out to the desired size, and the cubes later scaled by insertion into any larger sized substrate (FIGS. 17,18,19) for any use such as in rebuilding wild coral reefs, storage of coral specimens in a lab setting, bio-medical research, for sale to the aquarium hobbyist industry, etc.

This first method I invented, the Dimple Cube Method (DCM), was only partially successful in allowing certain species of soft coral cuttings 108 attach to a new substrate and grow out. The success rate varied according to species. Using the DCM, some species had a 0% attachment and survival rate, and some species had a 100% attachment and survival rate. Most fell somewhere in between.

Disadvantages of the Dimple Cube Method

Upon observation, I found that most types of soft coral cuttings 108 would not survive if part of their tissue was laying on the non toxic substrate (NTS) while healing and attaching. I hypothesized that most likely this was due to lack of water circulation (and likely lack of oxygen) underneath the soft coral cutting 108. This would lead to tissue necrosis on the underside of the cuttings, resulting in infection, tissue disintegration and death. The occluded area underneath turned black with a distinct sulfur smell of tissue decaying and left a distinct black mark in the bottom of the dimple. Soft corals have no stony (calcium carbonate) skeletons, only tiny support structures in their tissue called sclerites. So upon death, there would be nothing left except sometimes a small pile of sclerites

Because of the high mortality rate for some species using this method, I needed to find a way to have a higher attachment and survival rate for all corals. That led to the discovery of Method B, the Tunnel Cube Method (TCM) below.

Method B—High Volume Asexual Propagation Technique for Alcyonacean Soft Corals Using the TUNNEL Cube Method (TCM)

The object of this invention was to create a more reliable method to ATTACH individual juvenile soft coral cuttings 108 to a small cementitious cubed substrate without the high failure rate of the Dimple Cube Method (DCM) for some species of coral.

This second method, the Tunnel Cube Method (TCM), was successful in allowing many formerly difficult to attach species of soft corals to attach to a new substrate and grow out. It also allowed a higher survival rate and lower mortality rate on other coral species that were only partially successfully attached using the DCM.

Specifically this method allows very small soft coral cuttings 108 to become attached and grow out inside of the vertical tunnels of small cubes made of a cementitious non-toxic substrate (NTS) approximately 13 mm×13 mm×10 mm deep, which are efficiently stored in a lattice (Tunnel or Dimple Plates). The size of the tunnel could vary considerably depending on the species and design of the salt water system.

They key component of this discovery is the tunnel running vertically through the middle of each small cube. A fiberglass net 109 is placed under the cube to contain the soft coral cutting 108, and a more porous net 110 is placed over the top of it to prevent the coral from blowing out of the tunnel in the current. The soft coral fragment is completely contained on all sides but still has sufficient water and oxygen available on all sides, plus lighting from above, to encourage it to survive, and to attach. Given the right conditions, the coral will permanently attach itself to the side of the tunnel and begin to grow.

Each tunnel cube 105, containing one or more asexually produced soft coral cuttings 108 (of the same or different species) attached inside of the tunnel, could then be efficiently moved, shipped, or stored in it's high density eggcrate lattice framework (FIGS. 4a, 4b). The soft coral cuttings 108 could then be grown out in size, and the cubes later scaled up for insertion into any larger sized substrate for any use such as in rebuilding wild coral reefs, storage of coral specimens in a lab setting, bio-medical research, for sale to the aquarium hobbyist industry etc.

After successful attachment and stabilization of the soft coral cutting 108, the coral farmer can then choose from several options. The farmer can choose to ship the dimple or tunnel cube plate 107 as is, or to transfer individual cubes to larger substrates for further grow-out, or to plant individual coral cubes in the ocean for rebuilding a reef.

### Advantages of this Method

All of the advantages from the Dimple Cube Method apply to this method also.

However, the greatest advantage to the Tunnel Cube Method (TCM) over the dimple cube method, and all other methods that use a depression in the substrate to hold the soft coral cutting 108, is that many previously unsuccessfully attached species of soft corals are now able to be successfully attached to their initial substrate and allowed to grow out. Also the success rate of attachment for many soft coral cuttings increased dramatically, often to 100%. Mortality rates of soft coral cuttings are close to zero for nearly all species that attach using this method.

The cube method is the most efficient way to grow-out soft coral cuttings 108. At this density, one square meter of tank surface can hold up to about 5200 juvenile starter corals. Since each plate is a mono-culture, there is no problem with chemical burning or aggressive behavior between soft coral cuttings 108 or grow outs. Grow out plates can also be further arranged as to optimize tank space. If plates are placed in rows at a 45 degree angle to the surface of the water for grow out, one square meter of water surface can hold up to 7500 juvenile starter corals.

Most species have a very high attachment/low mortality rate with this method. Other species have a low attachment/low mortality rate with this method. In the latter case, the Dimple cube method (B) can be used as it provides for a tighter hold (due to the top net 110) on the soft coral cutting 108 while it is becoming attached. But many if not most soft corals will not tolerate such restrictions, so the tunnel cube method should be used (TCM).

While these two methods were very effective in propagating most soft corals, there was one other method that was developed, to take advantage of something else observed during natural reproduction. This led to Method C.

Method C—High Volume Asexual Propagation Technique for Corallimorpharian Soft Corals on Glass Plates 113.

Although Corallimorpharians are taxonomically more closely related to stony reef building corals (Scleractinians), the absence of a stony skeleton requires an asexual propagation method that is more similar to the Octocorallians.

The object of this invention was take advantage of a natural method of asexual soft coral reproduction that was observed in the saltwater systems, and to optimize the method to produce many more corals than they would normally produce even in nature.

This method allows newly produced juvenile soft corals 119 to grow on glass plates 113 in very high density.

Certain species of corals (Actinodiscus, Zoanthus, Discosoma, Amplexidiscus, Rhodactis, Dendronepthya genera) naturally propagate asexually from their pedal (foot) disks as they move along rocks at an indiscernible pace, often just a few millimeters per month. They leave traces of their pedal tissue which, once disconnected from the mother colony, quickly grows into a separate new coral.

The goal was to get the pedal disk 117 attached to some type of smooth substrate that would allow me to make clean minute cuts separating the pedal disk 117 into many sections. If during cutting, if any part of the coral is torn or crushed, the pedal disk 117 will easily get a bacterial or fungal infection causing a total loss of the tissue.

In this Glass Plate Method (GPM), corals are attached via their pedal discs directly to glass plates 113, without any intermediate rock or other substrate. Once firmly attached, the pedal disk 117 grows in size, forming an irregular, roughly spherical shape. When the cap and stalk is excised from the coral, the pedal disk 117 will grow into one new coral, still attached onto the glass plate 113.

The contiguous pedal disk 117 tissue of the coral will only produce one adult colony, regardless of the size of the pedal disk 117. However, by dividing the pedal disk 117 carefully, you can create very large quantities of tiny corals which will all grow into individual adults, still attached to the glass plate 113 (FIG. 15).

Glass plates 113 were chosen to grow the corals on, since they are very smooth, and present a hard backing when cutting on them with a scalpel. This would allow very precise, clean cuts without any damage to the coral tissue.

The challenge was getting the mother colony to attach to glass for the first time, without any other intermediate substrate between the glass and the corals' pedal disk 117. This was accomplished using several methods.

One method is to reduce the natural rock substrate connected to the pedal disk 117 as much as possible by grinding it away. This rock substrate can then be temporarily glued to the glass plate 113. The coral will naturally walk off of the rock over a few weeks or months time, onto the glass plate 113, seeking better conditions. You can then remove the rock and will have the coral attached directly to the glass plate 113. It is then ready for propagation or you can let it grow out further, growing more pedal tissue, before removing the cap and dividing the pedal tissue.

The advantage of the method is the number of corals produced asexually is far greater than if you let nature take it's course. Removing the cap and stalk of just one coral (FIG. 13) yielded 70 new juvenile corals after only a few weeks. Had the pedal disk not been artificially divided, it would have yielded only one new coral. FIG. 12 shows a drawing of an actual glass plate that started with about 12 adults that were then excised leaving their pedal disks. This one glass plate yielded around 600 new juvenile corals.

### BRIEF DESCRIPTION OF THE SEVERAL VIEWS OF THE DRAWING

FIG. 1 is a perspective view of a solid base cube manufactured by filling polystyrene eggcrate panels (often used as lighting diffusers) using a non-toxic proprietary cementitious substrate that was allowed to harden. Cell (cube) size in this example is ½ inch×½ inch×⅜ inch deep (12.7×12.7×9.5 mm) but other sizes may be used. Other materials could be used if they can allow the cubes to be removed and replaced easily in the eggcrate.

FIG. 2 is a perspective view of a dimpled cube. Same as FIG. 1 except indented with a small dimple to hold the soft coral cutting 108. Dimple size may vary according to the coral cutting size.

FIG. 3a is a top view of the dimple cube in FIG. 2 with a soft coral cutting 108.

FIG. 3b is a cross section view with soft coral cutting 108 (taken from center line) of the dimple cube.

FIG. 3c is a bottom view of the dimple cube.

FIG. 4a is a view from above of an empty dimple cube plate 104 comprised of a polystyrene eggcrate plate containing 117 dimpled cubes in FIG. 2. Size and quantity of the cubes could be varied according to need.

FIG. 4b is a perspective view of FIG. 4a empty dimple cube plate.

FIG. 5 is an exploded perspective view of a dimple cube plate assembly (DCPA) with soft coral cuttings 108 in each cube, including a dimple cube plate (FIG. 4a) and other materials 110, 111, 112 to contain the soft coral cutting 108 in the dimple 103 while allowing water and light to reach it.

FIG. 6 is an exploded view cross section side view of the dimple cube plate assembly (partial) with soft coral cutting 108 (DCPA) of FIG. 5.

FIG. 7 is a perspective view of a tunnel cube (TC) 105, created by applying a mold and filling polystyrene eggcrate panels using a non-toxic proprietary cementitious substrate that was allowed to harden. Cell (cube) size in this example is about ½ inch×½ inch×⅜ inch deep (12.7×12.7×9.5 mm) but other similar sizes may be used. Other materials could be used if they have similar density and can allow the cubes to be removed and replaced easily. Tunnel size may vary according the soft coral cutting 108 size or the species being propagated.

FIG. 8a is a top view of the tunnel cube in FIG. 7 with a soft coral cutting 108.

FIG. 8b is a cross section view with soft coral cutting 108 (taken from center line) of the tunnel cube.

FIG. 8c is a bottom view of the tunnel cube.

FIG. 9a is a view from above of an empty tunnel cube plate (TCP) 104 comprised of a polystyrene eggcrate plate containing 117 tunneled cubes in FIG. 7. Size and quantity of the cubes could be varied according to need.

FIG. 9b is a perspective view of a tunnel cube plate in FIG. 9a

FIG. 10 is an exploded perspective view of a tunnel cube plate assembly (TCPA) including TCP 107 and other components 109, 110, 111 to contain the soft coral cutting 108 in the tunnel while allowing water and light to reach the soft coral cutting 108 from all sides including underneath it.

FIG. 11 is an exploded cross section view (partial) of the tunnel cube plate assembly with soft coral cutting 108 (DCPA) of FIG. 9

FIG. 12 is a glass plate 113 with undivided pedal disks 117 remaining after adult coral tentacle and stalk cut off (taken from actual photograph).

FIG. 13 is a magnified view of one pedal disk on glass plate 113 pedal disk showing the lines of the initial cuts with scalpel on day one. (taken from actual photograph).

FIG. 14 is a glass plate 113 pedal disk closeup taken 3 days after first cuts 118 of pedal disks with scalpel showing separations of the pedal disks (taken from actual photograph).

FIG. 15 is a glass plate 113 pedal disk closeup taken 23 days old Juvenile Corals 119 showing growth of stalks and tentacles from pedal disk cutting (taken from actual photograph).

FIG. 16 is a partial view of glass plate 113 adults soft corals grown out and completely covering the plate (taken from actual photograph) after about 7 months.

FIG. 17 is an example of Universal Artificial Rock (UAR) with a female cube depressions. Dimple or tunnel cubes may be inserted into female cube for grow-out or sale. This UAR is made of a cementitious material but may be any shape, size, or composition.

FIG. 18 Universal Artificial Rock (UAR) male with protruding cube.

FIG. 19 is Universal Combo Rock similar to FIG. 17 except with three female cube depressions for dimple or tunnel cubes. Number of female depressions can vary.

FIG. 20 is a perspective view of Polystyrene eggcrate. Standard eggcrate polystyrene material (often used as lighting diffusers, typically about 14 mm squared on top) is typically sold at home improvement stores in 2′×4′ sheets. Cell (cube) size: ½ inch×½ inch×⅜ inch deep (12.7×12.7×9.5 mm). Other materials could be used if they have similar density and can allow the cubes to be removed and replaced easily.

FIG. 21 Configuration of rubber bands 112 holding the TCPA and DCPA. Rubber bands are used to isolate each cell to prevent soft coral cuttings 108 from moving do a different cell or being lost.

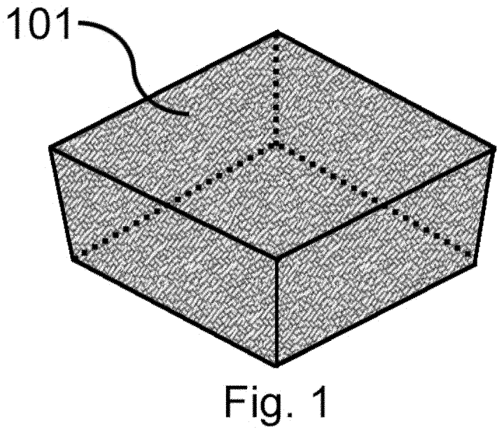

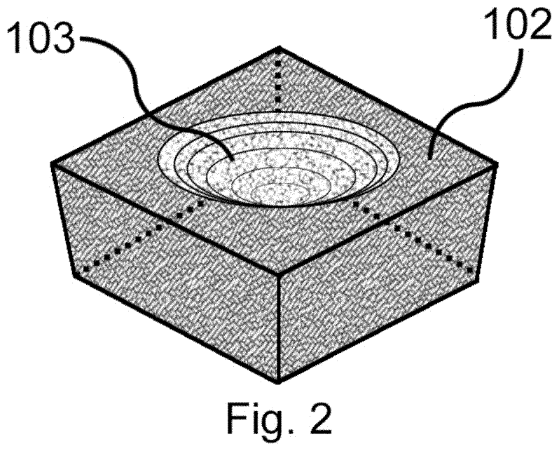

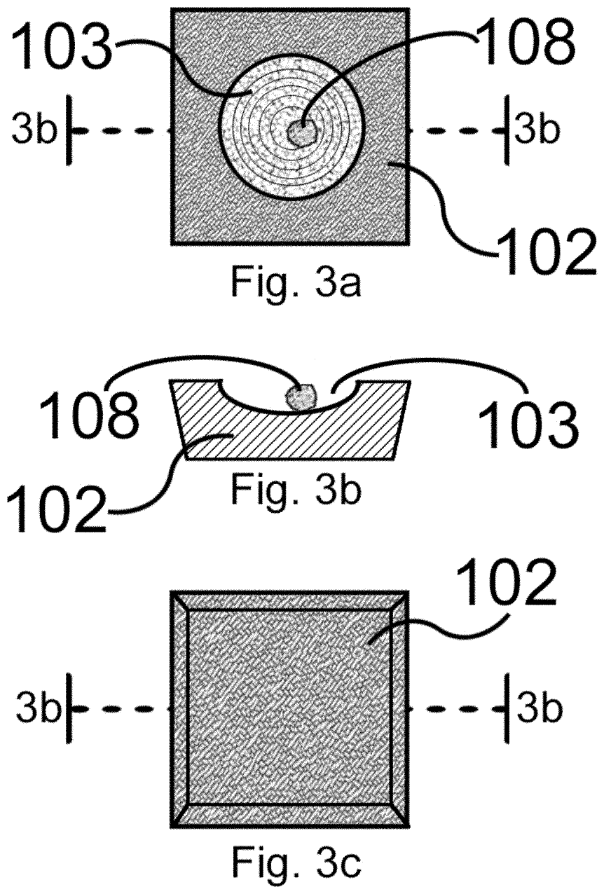

### DEFINITIONS

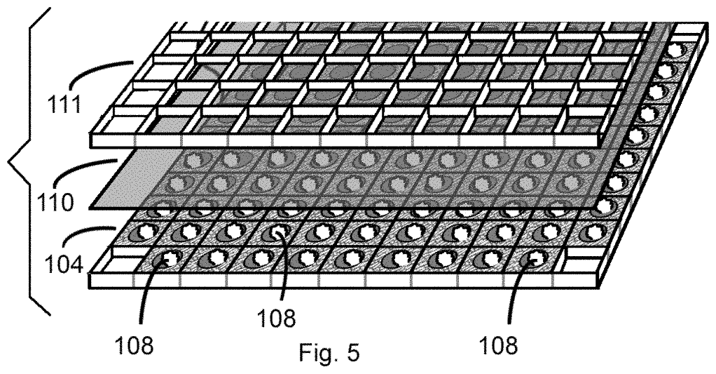

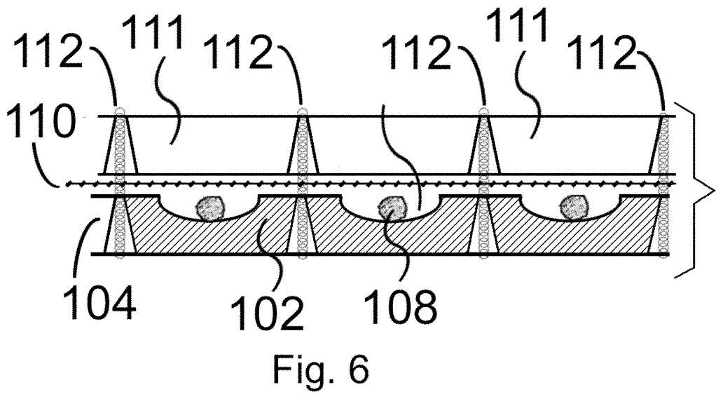

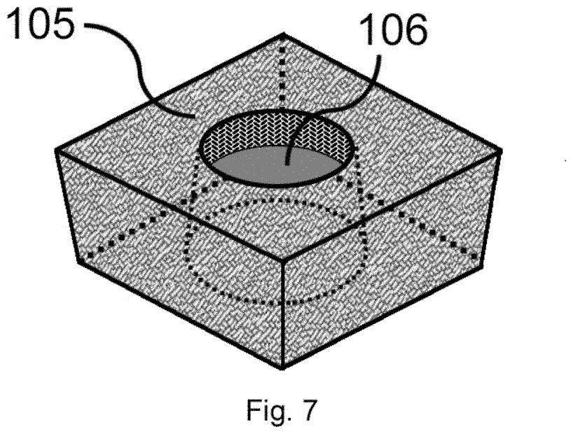

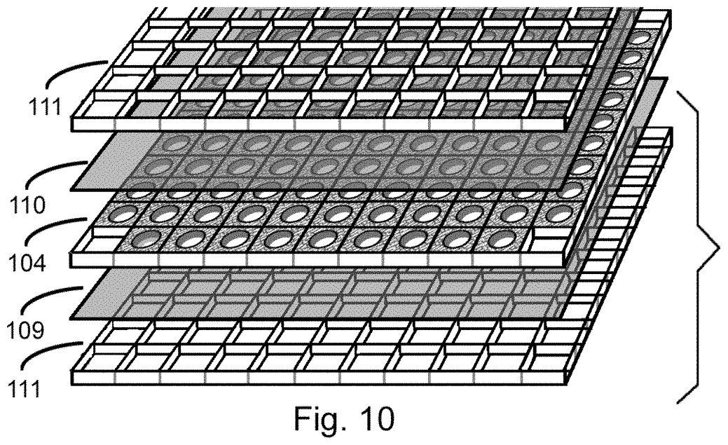

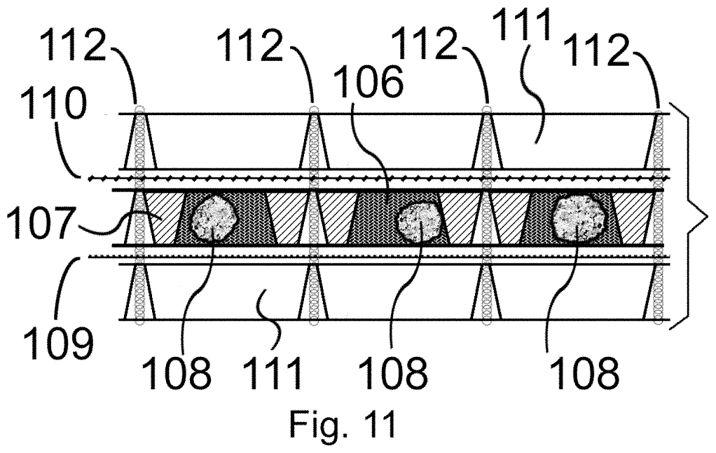

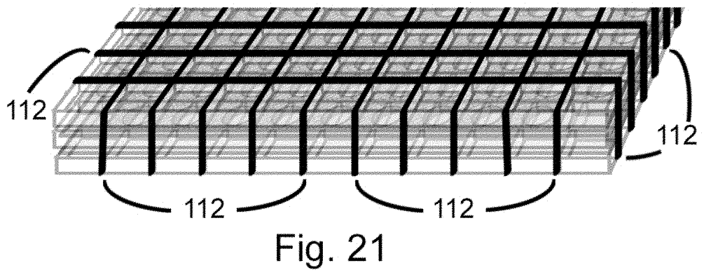

NTS. A Non-Toxic Substrate (cementitious) that can be poured into molds and hardens within a short period of time. Cementitious material must be inert when cured to avoid caustic burns to the corals and also not affect the pH of the coral water system. Other materials may be used as long as they are non-toxic to the coral. The material we use for this is proprietary and has no apparently toxicity to corals even when newly poured before setting.
101 Cubes. Three dimensional cubes made simply by using a standard eggcrate plates and filling it completely with a non toxic cementitious substrate NTS that is then hardened and cured. The result is a solid cube with a flat surface on top and bottom, and beveled sides to fit within the eggcrate matrix. The top surface will be slightly larger than the bottom surface, to stop the cube from slipped through the eggcrate matrix. Cubes may either be solid, dimpled, or tunneled. The size of the cubes must be standardized so that they are interchangeable, but any size would work. After the NTS sets up (30 minutes or so depending on factors such as the amount of water in the mix, ambient temperature and humidity), the plate can be put into the system where the corals will grow out, to cure further.
102 DC (Dimpled Cubes). A solid cube with a small dimple in the top, which holds the soft coral cutting 108 below the line of the netting above, to avoid compressing or damaging the soft coral cutting.
103 Dimple. Small depression in the surface of the substrate, to hold the coral cutting while it attaches.
104 DCP (Dimple Cube Plate). An eggcrate matrix plate containing 117 (or any other number of) dimple cubes 102.
DCPA (Dimple Cube Plate Assembly). (FIGS. 5,6) An assembly containing the dimple cube plate 104, soft coral cutting 108 in each dimple, covered by a polyester (or similar material) net 110 and an empty eggcrate plate on top aligned with the DCP. The assembly is held in place by rubber bands 112 along the edges of all cubes.
DCM (Dimple Cube Method). The procedure for attaching and growing soft coral cuttings 108 using the Dimple Cube Assembly
105 Tunnel Cubes (TC). Cubes having a tunnel (hole) in the center running vertically from top to bottom. Hole shape may be cylindrical or irregular. The tunnel is wider at the bottom than the top of the cube. Water can flow through the cube either from above or below, alleviating the anoxic zones that can cause a high mortality rate among soft coral cuttings 108. Sunlight or artificial light shines through the hole from above, attracting the coral cutting to grow up the side of the cube and attach to the cube instead of the netting 109 underneath. Tunnel cubes 105 can be made out of any non-toxic (to the corals) materials including but not limited to cement, plastic, rock, or noncorrosive metal. It's the shape of the cube that is most important, not the material as long as the material allows for strong attachment of the coral to the material. Rough and slightly porous surfaces seem to provide the best adhesion.
106 Tunnel. A vertical tube running through the center of the cube, containing the soft coral cutting(s) and providing the permanent point of attachment. The tunnel eliminates any dead spots which can cause the cutting to die, and allows free flow of water from above and below the cube, plus light from above. As the cutting expands, it will fill up the tunnel completely, firmly anchoring the coral, which will continue growing out the top of the tunnel.
107 TCP (Tunnel Cube Plates). An eggcrate matrix plate containing 117 (or any other number of) tunnel cubes. 105
TCPA (Tunnel Cube Plate Assembly). (FIGS. 10, 11). An assembly containing the tunnel cube plate 107, with a soft coral cutting 108 in each tunnel, covered by a net on both top 110 and bottom 109, and an empty eggcrate plates on top and bottom, both aligned with the TCP. The assembly is held in place by rubber bands 112 along the edges of all cubes. (FIG. 21)
TCM (Tunnel Cube Method). The procedure for attaching and growing soft coral cuttings 108 using the Tunnel Cube Assembly
108 Soft Coral Cutting. Coral cuttings from the Order Octocorallia (Alcyonacea) which makeup a large percentage of coral reefs and are essential to the biodiversity of reefs. Unlike stony corals (hexacorallians), soft corals have no stony (calcium carbonate) skeletons, only tiny support structures in their tissue called sclerites.
109 Fiberglass screening or netting. Commonly used as window screening.
110 Polyester green net. Hole size as large as possible without allowing the coral cuttings to escape.
111 Polystyrene eggcrate. Standard eggcrate polystyrene material (often used as lighting diffusers, typically about 14 mm squared on top) typically sold at home improvement stores in 2′×4′ sheets. Cell (cube) size: ½ inch×½ inch×⅜ inch deep (12.7×12.7×9.5 mm). Other materials could be used if they have similar density and can allow the cubes to be removed and replaced easily.

Eggcrate plates. Made of standard eggcrate cut into sheets of 11×11 rows yielding 121 cells. Plates could be cut into any size or shape depending upon needs and system configuration. To create plates filled with cubes, empty eggcrate plates are placed over various molds and NTS is poured into these and allowed to solidify and cure. Corner cubes may be removed to allow for hanging in the tank yielding 117 usable cube spaces.

112 Rubber bands. Primarily size #35, ⅛ inch wide, was used to secure the assemblies and not obstruct the cube openings.

113 Glass plate. Adult soft corals are attached directly to the glass plates 113, with minimal substrate between the coral and glass. Coral will migrate to the glass only after a period of time. Substrate other than glass is eliminated completely. Glass will enable extremely precise cutting (separation) of the pedal (foot) tissue into much smaller sizes that are all still attached to the glass. Such precision avoids the damaging of coral tissues which often lead to bacterial, viral, or fungal infections, tissue necrosis and coral death. Plates are reinforced against breakage, and may be 216 mm×279 mm or any other convenient size.

114 Cube female opening. The opening in a UAR that accepts either a solid, dimple, or tunnel cube.

115 Cube male on UAR protruding. The cube-shaped projection from a UAR allowing it to be inserted into an eggcrate matrix, or a larger UAR female receptor 116.

116 Female Receptor for Male Cubes. An opening in a UAR to accept a cube male 115.

117 Coral Pedal Disk foot. The portion of a soft coral connected to a stalk on one end, that attaches to a substrate on the other end.

118 Coral Pedal Disk foot cuts. Precise cuts made in the pedal disk to separate the tissue completely, allowing the pedal disk tissue to morph into a fully formed juvenile soft coral.

119 Juvenile soft corals. Progeny of asexual reproduction of adult soft corals.

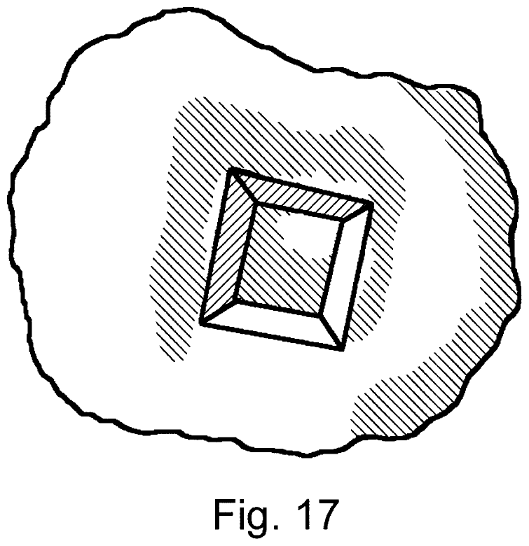

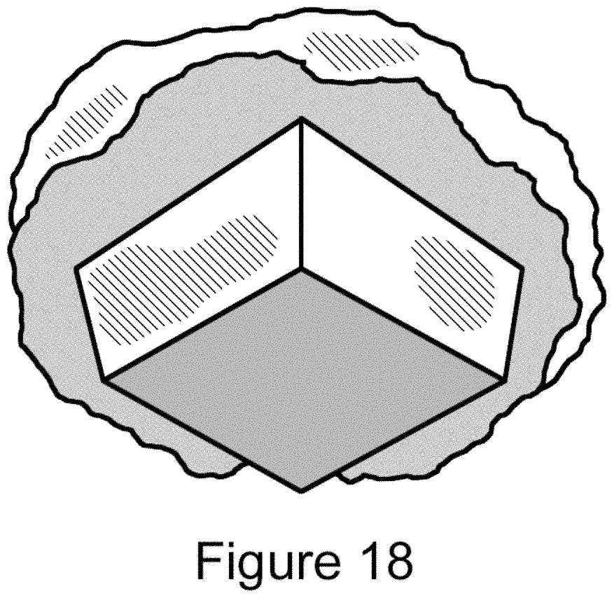

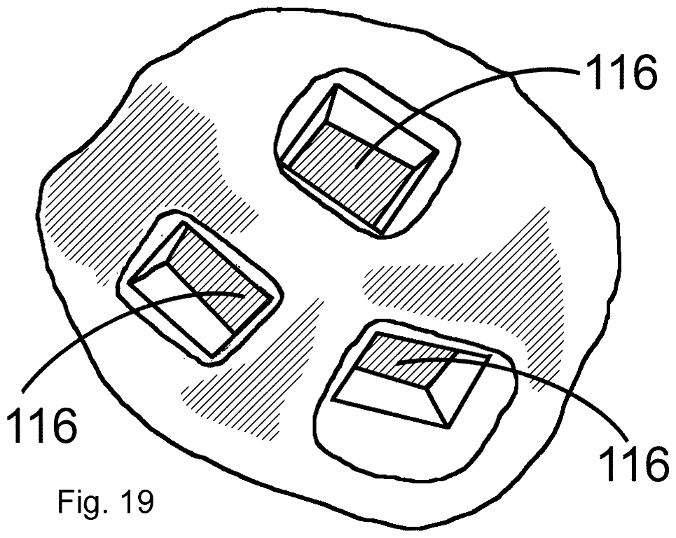

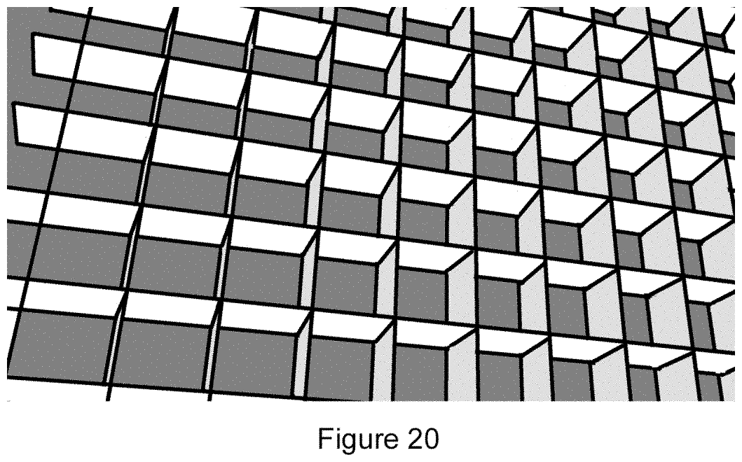

UAR (Universal Artificial Rock). (FIGS. 17, 18). Made with NTS, this artificial rock may or may not have a protruding cube on the bottom to slot into the eggcrate and has one or more inverted cube openings on top or on the sides to accept any cubed corals. UARs are made using latex molds but could also possibly be 3D printed.

Combo Rock (CRX)— (FIG. 19) A universal artificial rock with openings for more than one cube. Allows either the same coral species to be placed on a larger rock, or different compatible species to be placed on the larger rock. When replanting a reef, this should cut down on the manual labor when placing the corals in the ocean. For reef replanting, the shape of the rock could be modified, to enhance secure placement of the new coral on the reef. For aquarium hobbyists, this would allow the purchase of one rock with two or more corals on it. Choices of corals would be complimentary for color, shape, and compatibility, to optimize them aesthetically.

### DETAILED DESCRIPTION OF THE INVENTION

Method A—High Volume Asexual Propagation Technique for Octocorallian Soft Corals Using the Dimple Cube 102 Method (DCM)

Originally it was attempted to grow soft coral cuttings 108 individually on natural calcium carbonate rocks, wrapping the large cuttings with a net and rubber banding them. Success was limited with some species, but most coral species were not successfully propagated at all. Mortality rates were high and the method was labor and space intensive.

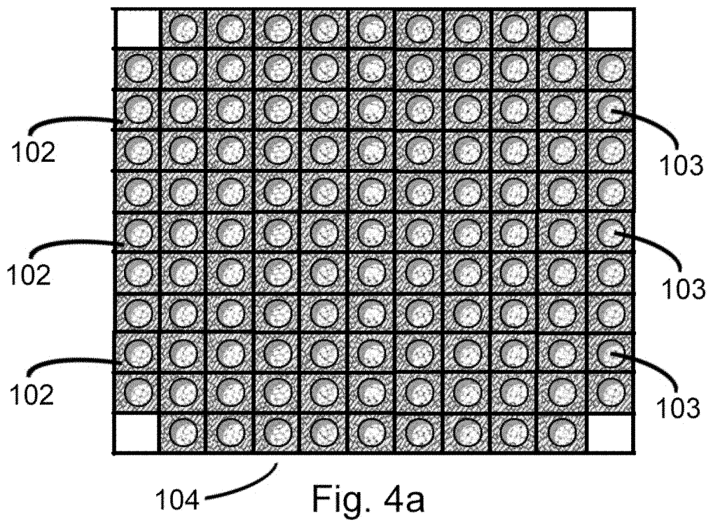

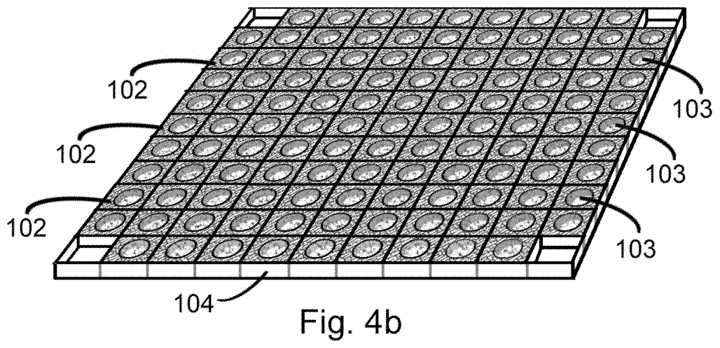

After long deliberation and much experimentation, cubes with small depressions called dimple cubes 102 were created. The dimple cubes 102 were arrayed on a polystyrene eggcrate 111 plate which together was called a dimple cube plate (DCP) 104. (FIG. 4b).

Dimple Cube Method:

The method using dimple cubes to propagate soft corals evolved over time. First a healthy acclimated colony of the soft coral is removed from the saltwater tank and parts of it's tissue (usually the capitulum or cap) is carefully excised. Often only the outer ring of the capitulum is removed (particularly with Sarcophyton sp.), leaving the tissue over the stalk in place. This aided in the quick recovery and regrowing of the capitulum.

It's extremely important to handle the coral with care and not crush any tissue, which will likely result in a mortal infection.

On a clean smooth cutting board, with a sharp scalpel, carefully slice the selected soft coral tissues into small pieces compatible with the dimple size. The size and shape of the cuttings are species dependent. It's important to be careful not to crush the tissue in any way. Clean cuts are essential to success.

Most cuttings require leaving them in a container of saltwater for 20 minutes or so, to allow the cuttings to “demucous”. This thick mucous is poured off and discarded, often several times. Excessive mucous on the cuttings will often lead to infections, as it provides nutrients for bacterial growth.

It's recommended that any corals held outside of water for more than a few minutes should be periodically sprayed with saltwater of the same tank water it came out of, to keep it from desiccating.

Immediately before use, rinse soft coral cuttings 108 in same saltwater one final time, removing any remaining mucosal debris. With forceps or an eggbaster, gently install one soft coral piece per dimple, without crushing the cutting. Usually there is one cutting per dimple, but more could be used, or different compatible species could also possibly be combined in one cube.

After all of the dimples are filled, flexible polyester net 110 was applied on top of the dimple cube plate, and then an empty eggrate plate on top of the net, aligned perfectly with the DCP. Rubber band 112 the plates along each side of the cubes both ways (FIG. 21) so as not to block current or light. The hole size of the net 110 should be smaller than the cuts but as large as possible for maximum water flow and light.

The completed dimple cube plate assembly DCPA (FIG. 5) can then be placed in the saltwater system with a fairly high water flow area with moderate lighting. Its imperative that there is no detritus circulating in the system that could clog up the nets 109, 110.

Attachment time is species dependent, and takes from a week to several months. Coral cuttings could be observed from above without disturbing the plates. That may give a good indication as to when the plates are ready to be pulled and the top net 109 removed.

Overall the dimple cube method was a huge improvement over the standard method of placing a coral cutting on a rock and wrapping it with a net and rubber bands.

Problems with the Dimple Cube Method

While this dimple cube method worked well for certain genera of hardy soft corals (Xenia, Cespitularia, Clavularia, some Sarcophyton, Anthellia), unfortunately it did not work for most other species of soft coral. Certainly there are probably other soft coral species that could be effectively propagated using this method. Actually many invertebrates such as sponges could also be propagated using this method.

Corals from many important genera such Sinularia, some Sarcophyton, Palythoa, Discosoma, Zooanthus, Heteroxenia, Lobophyton, and Actinodiscus had a very high mortality rate with the dimple cube method.

It was determined that most types of soft coral cuttings 108 would not survive if part of their tissue was laying on the NTS while healing and attaching. Most likely this was due to lack of water circulation (and likely lack of oxygen) underneath the soft coral cutting 108. This would lead to tissue necrosis on the underside of the cuttings, resulting in partial disintegration and death. The occluded area underneath turned black with a distinct sulfur smell of tissue decaying. Soft corals have no stony (calcium carbonate) skeletons, only tiny support structures in their tissue called sclerites. So upon death, there would be nothing left except sometimes a small pile of sclerites

Several modifications were tried, such as varying the water flow direction and intensity, lighting intensity and type, size of the net openings, and even changing water parameters such as temperature and alkalinity. None of these significantly changed the success rate of soft coral cutting 108 survival and attachment. They continued to decay, starting first with the underside that was in contact with the NTS.

What was needed was a propagation method that didn't restrict the access of oxygen and water flow to all sides of the cutting, while it was in the process of healing and attaching to the NTS.

The Solution

Finally I came up with the idea to create a vertical tunnel in the center of each individual cube, allowing water to circulate from above AND below the cutting. So the cutting would have access to water around it's entire surface all of the time. It was hoped that this would solve the problem of having an occluded area which often was leading to tissue necrosis.

The obvious concern was that the soft coral cutting 108 would simply fall to the bottom of the tunnel, land on and attach to the bottom fiberglass netting 109. This wouldn't help at all since it was the attachment to the NTS cube, not the netting 109, that was required for success.

It was decided to try it anyway, to see what would happen. And I was quite surprised by the results!

Method B—High Volume Asexual Propagation Technique for Octocorallian Soft Corals Using the Tunnel Cube Method (TCM)

Tunnel Cube Method.

Getting stony corals to asexually propagate by fragmentation is simple. Methods commonly used include using cyanoacrylate glue or waterproof plastic epoxy putty to attach it to a substrate. Getting soft corals to asexually propagate by fragmentation is like getting jello to attach to rock.

Commonly used methods of asexual hard and soft coral propagation also are highly inefficient, time consuming, expensive, and require a large amount of space. The tunnel cube method substantially improves the propagation process in all respects.

Most types of soft coral cuttings 108 will not survive if part of their tissue is against the substrate while healing and attaching. Lack of water circulation (and likely oxygen) underneath the soft coral cutting 108 will often lead to tissue necrosis, disintegration and death. The occluded area turns black with a distinct sulfur smell, leaving only a small pile of sclerites.

The Tunnel Cube Method (TCM) solves that problem by allowing water flow around all outer parts of the coral tissue while it is healing and attaching to the NTS. Having a fiberglass screen 109 below and polyester screen 110 above the cutting allows flow to reach all parts of the soft coral cutting 108 as long as it is smaller than the tunnel when expanded back to natural size. Most soft corals shrink substantially when disturbed and cut, and then expand back to normal size after a short period of time. It's important that the expanded cutting size is smaller than the tunnel size. Otherwise it will create a dead zone, possibly resulting in necrosis.

Initially it was expected that the cutting would attach to the bottom net only 109, as gravity pulled it to the netting. What was found after experimenting was the coral cutting 108 will attach to both the bottom screening and the side wall of the tunnel. If left for 30-60 days or more (again species dependent), the attachment on the sidewall was strong enough that the screening could be peeled away, leaving the soft coral cutting 108 attached to the tunnel sides only, which was what was desired. This was quite unexpected. In some cases, the cutting had to be sliced away from the netting 109 as it was peeled back, with excellent results.

However in many cases, the coral cuttings had already started to climb the sides of the tunnel and there was no attachment at all to the bottom net 109. This was unexpected, and nature had solved the problem of net attachment for us.

Tunnel cubes 105 are just a variation on the dimple cubes 102, except they allow water flow from both above and below the cube plates. But the decrease in mortality rates from just this one simple change was astonishing. Whereas the rate of dimple cube 102 mortality was often 100% for many soft coral species, the tunnel cube 105 method produced a 100% success rate for many of those same corals, and a high 80 plus percentage rate for many others. This was a major step forward, and allowed the growth of a virtually unlimited number of many species of juvenile soft corals 119, limited only by available tank space.

It's very important to make sure the tunnel becomes wider as it goes from top to bottom. Otherwise the soft coral cutting 108 may get lodged in the tunnel before reaching the lower net 109, creating an anoxic zone between the cutting and the wall of the tunnel, which can lead to necrosis. The cutting should be able to move around freely at the bottom of the tunnel. Later the cutting will expand, heal, and then attach and grow to the side of the tunnel, eventually coming out of the top of the tunnel.

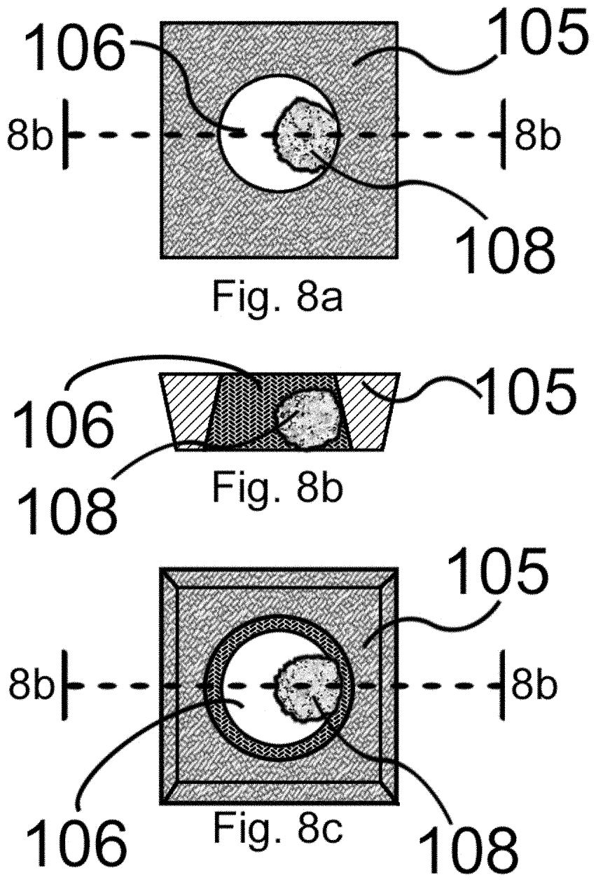

In reviewing the cross section of the cubes (FIG. 8), note that the bottom tunnel angles out and makes the space larger than the top opening. This aids in choosing the soft coral cutting 108 size. If the soft coral cutting 108 fits through the top opening, then it will have more room to expand when it settles on the net 109 at the bottom, resulting in more water circulation and a better chance of attachment. Also the angle provides some shading which the coral cutting may want to avoid, and likely further encourages it to attach to the wall and move up it towards the light.

Some soft corals do well in the simpler dimple cube 102 setup, while most definitely require the tunnel cube 105 setup. It's very much species dependent and we learned which species are successful in each method by extensive trial and error.

The First Success of the TCM

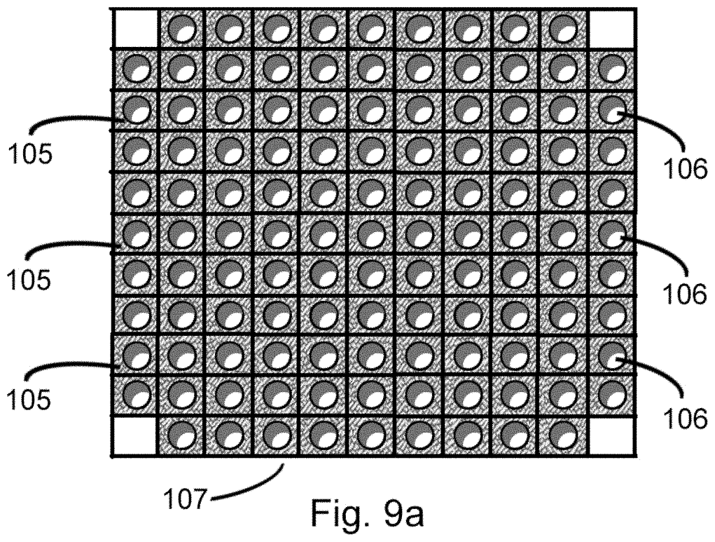

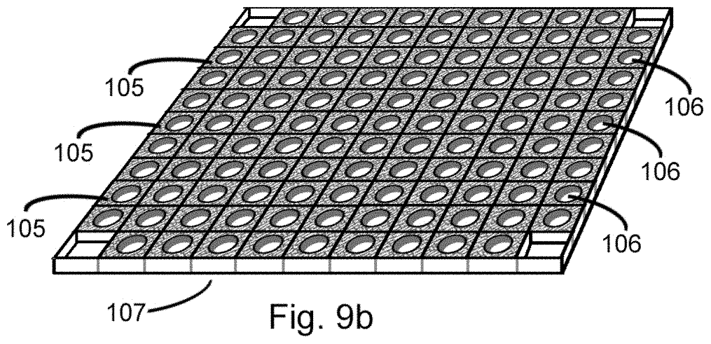

The TCP (FIG. 9a) was prepared, using healthy freshly cut Sarcophyton glaucum cuttings from a soft coral commonly called Fluorescent Green Leather (FGL), which are a very popular coral in the aquarium industry. Each tunnel hole was filled in every cube with a cutting. The TCPA (FIG. 10) was completed and placed in a dedicated system which had no detritus that might settle into the tunnel holes and block the water flow in the nets 109, 110. Water flow over and under the TCPA was adjusted as strongly as possible but without rolling the soft coral cuttings 108 around in their holes.

After 21 days, the TCPA was removed from the tank, disassembled, and the staff were very surprised to see 100% survival and 100% attachment! An excited notation was made in the daily log book.

The most amazing and unexpected part was that the soft coral cuttings 108 had attached to the bottom net 109 and the side of the Cube tunnel. The netting could simply be peeled away from the bottom of the plate and the soft coral cutting 108 would still be attached to the side of the cube. In fact many of the cuttings were only attached to the side of the cube and not the net 109.

Apparently, the cuttings “walk” towards the light above, at a rate much too slow to detect visually. But after unnetting them, after a holding period, all of the soft coral cuttings moved up the side of the tunnel and eventually filled in the tunnel completely as they grew in size. This makes sense as they seek light for the zooxanthellae in their algae.

The TCM was tried on many types of soft corals (Sinularia, Sarcophyton, Palythoa, Discosoma, Zooanthus, and Actinodiscus etc) that hadn't been successfully propagated using the dimple cube method, and the staff members were very surprised to see that all of them had a survival rate of over 90%, with most species at 100% survival rate! Attachment rates varied from about 40% to 100%. Sometimes attachment was only partial and the coral would dislodge as the TCPA was disassembled, so on the next tunnel cube plates, staff would increase the amount of time the plate was left before unnetting. Sometimes a cutting would simply disappear for unknown reasons.

For months a dated logbook was kept recording the details of each TCPA, noting it's survival rate, attachment rate, and length of time it was in the TCPA before unnetting 109, 110. Attachment rates varied according to the species and care of preparation, and probably other unknown factors. Each time a plate was unnetted, it was noted adjustments were made on some parameter (for the same species) such as size of the cutting, length of time netted, water flow over and under the plate, and amount of lighting over the tunnel cube plates TCP.

Fortunately if a soft coral cutting 108 survived but didn't attach, it would be collected and attachment attempted again, often with success. These were named “retreads”. The attachment rate of retreads was usually lower, probably because their surfaces were healed and not freshly cut. But it was found that if left in place for 60-90 days or more, most of the retreads would finally attach to the tunnel cubes 105. Also if retreads were re-cut, it aided in attachment.

Following are a few examples of experimental Tunnel Cube Plates 107 results. (Corals that were attached only to bottom net 109 were not counted as attached).

Lab Experimental Conditions

The experiments were done in a lab setting, in one of sixteen 1200 liter closed loop saltwater systems, using reverse osmosis treated artificial mix saltwater (Coralife, Seachem, Instant Ocean brands), under 400 watt (450 nm wavelength) metal halide bulbs and VHO fluorescent bulbs (250-400 nm wavelength) for lighting. Calcium reactors were used to maintain alkalinity and large high flow protein skimmers (foam fractionators) were used to remove dissolved and particulate waste. Water flow in the systems was maintained using a variety of Eheim pumps on timers.

Results were taken from the Daily Log book. Coral cuttings that survived but didn't attach (retreads) were tried again on other plates.

TABLE 1
Tunnel Cube 105 Trials testing for Survival and Attachment Rates

| | Duration Soft Coral | Survival | Attachment |
|---|---|---|---|
| | Cuttings left in | Rate | Rate |
| Soft Coral Name | tunnel cube | of Soft | (compared to |
| Order Octocorallia | assembly TCPA | Coral | original number |
| (Alcyonacea) | (days) | Cuttings | of coral cuttings) |
| Green Star Polyps | 20 d | 100% | 98% |
| Pachyclavularia | | | |
| viridis | | | |
| Fluorescent Green | 8 d | 100% | 90% |
| Leather | | | |
| Sarcophyton sp. | | | |
| Todd's Palythoa | 50 d | 100% | 84% |
| grandis | | | |
| Blue Xenia sp. | 18 d | 98% | 98% |
| Green Discosoma | 32 d | 88% | 71% |
| sp. | | | |
| Rhodactis sp. | 35 d | 68% | 44% |
| Fluorescent Green | 20 d | 100% | 100% |
| Sinularia sp. | | | |
| Bright Green | 27 d | 90% | 88% |
| Zoanthus sp. | | | |
| Capnella sp. | 20 d | 100% | 89% |
| Yellow Fiji Leather | 30 d | 97% | 94% |
| Sarcophyton sp. | | | |
| Cespitularia sp. | 20 d | 95% | 93% |
| Blue | 19 d | 100% | 98% |
| Protopalythoa sp. | | | |
| Striped Xenia sp. | 5 d | 100% | 100% |
| Waving Hand | 21 d | 100% | 98% |
| Anthellia sp. | | | |
| Pink Zooanthus | 40 d | 27% | 13% |
| sp. | | | |
| Fuzzy Green | 16 d | 100% | 100% |
| Sinularia sp. | | | |
| Fluorescent Green | 16 d | 98% | 90% |
| Leather + | | | |
| Zooanthus sp. | | | |
| Same hole. | | | |
| Elephant Ear | 43 d | 75% | 50% |
| Amplexidiscus sp. | | | |
| Clove Polyps | 43 d | 92% | 90% |
| Clavularia sp. | | | |
| White tipped | 27 d | 100% | 94% |
| Sarcophyton | | | |
| trocheliophorum | | | |
| Red Actinodiscus | 19 d | 100% | 97% |
| sp. | | | |
| Blue Actinodiscus | 19 d | 98% | 93% |
| sp. | | | |
| Maroon Frilly | 27 d | 88% | 70% |
| Discosoma sp. | | | |
| Green-striped | 33 d | 68% | 48% |
| Actinodiscus sp. | | | |
| Watermelon | 28 d | 10% | 10% |
| Actinodiscus sp. | | | |
| Green Galaxia sp. | 22 d | 100% | 100% |
| Chocolate Leather | 28 d | 99% | 94% |
| Sarcophyton sp. | | | |
| Green Pink striped | 42 d | 100% | 82% |
| Pachyclavularia | | | |
| sp. | | | |
| Blue Heteroxenia | 16 d | 100% | 90% |
| sp. | | | |
| Green Palythoa sp. | 24 d | 100% | 99% |
| Green Frilly | 23 d | 100% | 100% |
| Mushroom | | | |
| Discosoma sp. | | | |
| Green Zoanthus | 28 d | 82% | 94% |
| sp. | | | |
| Blue Palythoa sp. | 23 d | 100% | 100% |
| Frilly Brown/Green | 22 d | 100% | 98% |
| Discosoma sp. | | | |
| Orange Zoanthus | 38 d | 88% | 82% |
| sp. | | | |
| Purple/Red | 29 d | 90% | 90% |
| DotsActinodiscus | | | |
| sp. | | | |
| Xenia umbellata | 24 d | 99% | 99% |
| Yellow striped | 23 d | 100% | 99% |
| Xenia | | | |
| macrospiculata | | | |
| Xenia elongata | 23 d | 99% | 99% |
| Orange Ricordia | 32 d | 77% | 33% |
| florida | | | |
| Finger Leather | 29 d | 99% | 99% |
| Lobophyton sp. | | | |

Soft Coral Cuttings 108 Preparation:

As with dimpled cubes, the initial soft coral cuttings 108 preparation is the same. The only difference is the size of the coral cutting should be determined by the tunnel size, rather than the dimple size.

With a clean, sharp scalpel, on a clean smooth cutting board, carefully cut the soft coral into small cuttings, the size and shapes being species dependent. Being careful not to crush the tissue or subject it to any extreme environmental conditions is important. Don't allow it to dry out.

Immediately after dissection, place soft coral cuttings 108 into a small container allowing them demucus for about 20 minutes. Most cuttings will generate copious amounts of slime (mucus). Allow them to do this and then gently rinse the mucus away with aged salt water, often multiple times. Eventually they will quit sliming up and be ready for installation into a plate. Don't pour the mucous into the water system as some of it may be toxic to other corals.

It's recommended that any corals held outside of water for more than a few minutes should be periodically sprayed with saltwater of the same tank water it came out of, to keep it from desiccating

Tunnel Cubes Plate Assembly (TCPA) (FIG. 10) Preparation:

Ideally start with a tunnel cube plate that was soaked in aged saltwater for a period of time. However organic slime will build up on the plate if left too long and this will inhibit adhesion, so use non-biologically active salt water to age it. If applying coral cuttings inside of a dry tunnel cube plate, the cuttings may dry out quickly if not sprayed.

Before the soft coral cuttings are added, the fiberglass net 109 and an empty same-sized eggcrate plate must be attached to the bottom of the tunnel cube plate 107 to keep the cuttings from falling through when they are inserted. It's imperative to lineup both plates as to not impede water flow. Use temporary rubber bands to hold in place.

Immediately before their use, rinse soft coral cuttings 108 in same saltwater one final time, removing any remaining mucosal debris. With forceps or an eggbaster, gently install one soft coral cutting per tunnel, without crushing it. Usually there is one cutting per tunnel, but more could be used, or different compatible species could also possibly be combined in one cube. Adjust the size of the cuttings if more than one occupy a tunnel.

After all of the tunnels are filled, apply the flexible polyester net 110 on top of the plate, and then an empty eggrate plate on top of the net, aligned perfectly with the other two eggcrate plates. Hold the assembly together, remove the temporary rubber bands and then rubber band 112 the entire TCPA along each side of the cubes both ways (FIG. 21) so as not to block current or light.

The completed tunnel cube plate assembly TCPA (FIG. 10) can then be placed in the saltwater system with a fairly high water flow and with moderate lighting. Its imperative that there is no detritus circulating in the system that could clog up the nets 109, 110 leading to tissue death.

When assembled correctly, each tunnel cube will be sealed off from all other tunnel cubes, into individual compartments, with water flow from below and above, and lighting from above.

Attachment time is species dependent, and takes from a week to several months. Coral cuttings can be observed from above without disturbing the plates. Viewing without disturbing may give a good indication as to when the plates are ready to be pulled and disassembled.

When attachment appears to be complete, remove the TCPA from the tank, disassemble it, recover any coral cuttings that did not attach, and then place the now bare TCP back into the system in are area with moderate current. Allow them to grow out to the preferred size.

Some soft coral cuttings 108 may be alive but unattached (retreads). Collect these and use them in a new TP, but vary the conditions to improve chances of attachment such as less current, or leaving netted 109 110 for a longer period. Reslicing the surface may also enhance attachment.

It's important to monitor the plates during attachment. Sometimes if necrosis sets in or a fungus develops, it can overtake even healthy attached corals. So It's important to catch infections early and remove it. Often using an eggbaster to suction out the cube is enough to stop the spread.

The TCM could also possibly be used for attaching stony corals (Hexacorallia). Place one cutting per hole and allow the coral tissue to grow over the edge of the tunnel. Tunnel hole size might need to be adjusted depending on the species and diameter of the coral fragment.

Method C-High Volume Asexual Propagation of Corallimorpharians on Glass Plates 113.

Anatomically, Corallimorphian soft corals are different from the stony corals that they are taxonomically closely related to, since they do not possess a calcium carbonate skeleton. Most resemble a mushroom in shape, with a fleshy pedal disk 117 that anchors it to a substrate, an short or elongated midsection stalk that is similar to a tree trunk, and a wide oral disk (containing very short tentacles) that spreads out horizontally to gather light and nutrients, and sometimes catch prey.

The oral disk for Corallimorpharians seems generally not to be a good candidate for asexual reproduction. In experimenting with divided cuttings from the oral disk only, a 0% success rate of the coral attaching or even surviving was observed. We found that for most or these corals, leaving the entire cap intact was the key to survival, but not reproduction.

However it was observed that the pedal disk 117 or foot of the coral that was attached to the natural rock substrate, when divided, would create a new coral. Often it was observed that the coral had “walked” to a new portion of the rock over weeks or months, stringing behind it a portion of pedal disk 117 still attached to the substrate but without a stalk or oral disk. This would eventually separate from the primary adult coral, and then the pedal disk tissue would morph into a new stalk and cap quite rapidly. So clearly the foot propagated asexually and was a common method of reproduction in nature for these coral types.

In order to duplicate the natural asexual propagation method, a mother colony of Corallimorpharians was removed from the saltwater system, with the pedal disk firmly attached to a piece of live rock.

Initial attempts involved first cutting off the oral disk and stalk, and dividing the pedal disk 117 portion that was still attached to the natural live rock. The cutting was messy, difficult and incomplete due to the irregular surface of the porous rock. Also it was very difficult to cut small divisions in the pedal disk as cuts were attempted against the irregularly shaped rock. The scalpel become dull very quickly as it cut into the rock. The coral created copious amounts of slime because the process was stressful for it. The attempt was not successful, as the ragged edges of the crudely cut pedal disk soon became infected, spread, and the coral died.

Clearly pedal laceration was a viable method of asexual reproduction, but making the cuts on the natural rock wasn't going to work.

After analyzing the problem it was noted that the biggest issue was the corals became infected due to the tissue damage from the rough cutting on the rock as attempts were made to divide the pedal disk 117. Also cutting the pedal disk into the smaller sections desired was impractical, as the pedal disk wrapped around irregular sections of the substrate base rock.

A method was needed to better divide the pedal disk cleanly without tissue damage, and also cut into smaller sections. The coral needed to be attached to a very smooth hard surface, so that it could cut cleanly and in small sections without any tissue damage.

A glass plate were chosen as an ideal backing material candidate. It's non-porous, hard, and would make a perfect backing when cutting the pedal disk 117. Also the backside of the coral could be observed through the glass to verify adhesion. But there was no certainty that it was even possible for the soft coral pedal disk to attach to glass. I had never heard or read about corals attached directly to glass. Would the pedal disk 117 hold on?

Surprisingly it did. This is the procedure that was developed to accomplish the transfer of the soft coral from it's native rock to a glass plate:

Obtain a healthy specimen that is fully acclimated from your saltwater system. Corallimorphian soft corals are attached to a natural base rock by their pedal disk 117.

The initial process starts by artificially attaching the soft coral polyp to a glass plate 113. It's best to remove as much of the substrate that is connecting the pedal disk to the rock as possible without damaging the coral tissue in any way.

While there are several methods to remove rock, it was noted that the use a wet tile saw with a coarse diamond blade (or similar) easily removed as much of this base rock as possible, without cutting into the actual coral. The small portion of rock that remained attached to the coral was sawed into a flat plane, to improve adhesion of the coral rock to the glass plate.

The more rock base that was left, the longer it took for the coral to “walk off” of the rock and onto the glass. So it is important to minimize the amount of rock left attached. In some cases the coral will stay on the rock and not move off of it if too much rock is left.

After the base rock is removed, and is free of mucus, tamp the coral base rock with paper towels to remove as much of the water as possible from the rock. Use cyanoacrylate glue (aka “Crazy glue”) to attach the rock pieces to the glass plate 113. Allow a minute or two for the glue to setup, being careful not to get it on your fingers.

Place the glass plate 113 back into the saltwater system. The glue will immediately harden.

Glass plates 113 may be made out of glass or possibly any very smooth surface such as quartz, tile, waterproof laminates, polymethyl methacrylate (PMMA), any other non-porous hard material. It is recommended that you create a support structure on the back of the glass plate 113 in case they crack. Our glass plates had a eggcrate frame around the edges, secured with a marine grade silicon caulk.

Over a period of weeks or a few months, the corals will “walk” off of the base rocks and then become firmly attached to glass on it's own. The attachment to glass seems to be as secure as it is to rock. Often it will leave a “tail” of pedal disk 117 tissue on the base rock after it walks off. This can be saved.

Place the glass plate in the system at a 45 degree angle to horizontal. This will further motivate the coral to “walk” onto the glass and away from the small rocky base it was attached to. Very few of the corallimorpharians stay in one place unless penned in by other corals. They seem to be slowly always on the move, looking for better conditions and/or simply leaving their trails of pedal disk tissue for new offspring.

After the coral is firmly attached to the glass, remove the empty base rock piece and the cyanoacrylate when no more coral tissue is present on it and discard it. If tissue remains on the rock, repeat the above process on a different plate.

Place the glass plate 113 back in the system. After a few weeks or months, you should have a glass plate 113 with your adult soft corals firmly attached to it.

Allow ample time for the solid attachment of the pedal disk 117, for grow out, and to become comfortable and healthy on the glass plate 113. Ideally the soft corals will then spread out and grow large pedal discs with a large surface area in contact with the glass plates 113. The more pedal disk 117 in contact with the glass, the more divisions that can be made, and the more offspring that can be created.

Dividing the Pedal Disks

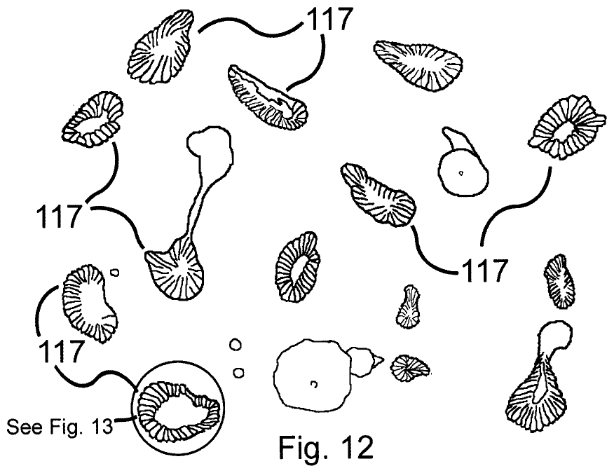

When the glass plate is ready, using a very sharp clean razor blade or scalpel, turn the plate over, let the oral disk and stalk hang down via gravity, and cleanly slice off the hanging oral disks and stalks intact, letting them fall into a container of clean tank water, leaving pedal disks 117 attached to glass. (FIG. 12) Ideally cut the stalk as close to the pedal disk 117 as possible, without cutting into the pedal disk 117 itself. Be careful not to tear or crush the tissue as this will invite infections later. Scissors should not be used as they crush the tissue. Do not divide the pedal disk 117 at this time. It needs to heal first.

Put the glass plate 113 with pedal discs back into the system, in relatively high water flow area and allow the plate to demucus and stabilize. Monitor it for fungal or bacterial infections. Within a few days, the pedal disk 117 tissue will begin heal and transform. Radial striations will become apparent in the pedal disk.

FIGS. 12-16 are drawings taken from actual photos of this method in progress.

After about a week, remove the glass plate 113 from the saltwater system. Under 10× magnification, observe the interior striations on the pedal discs. The pedal disks are ready to be divided. Waiting too long to divide the pedal disks will result in it morphing into one large single adult, precluding it's separation.

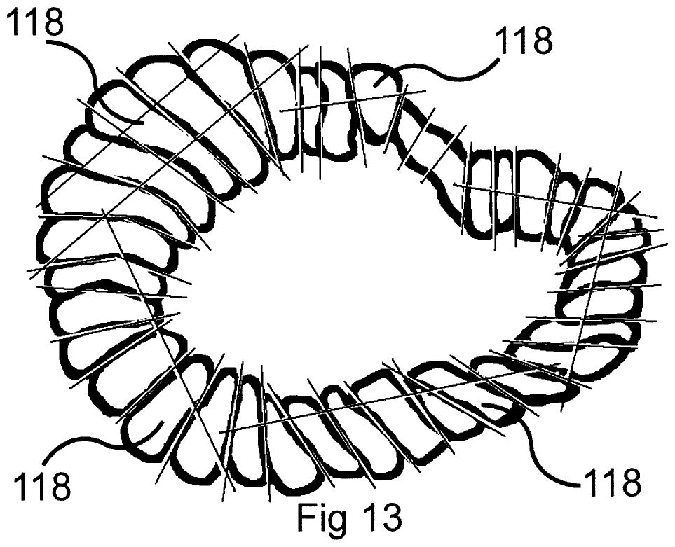

Using a new clean standard single edge razor blade, cut the pedal discs along the striations all the way down to the glass plate 113. It's important to separate the tissue completely, not leaving any tissue connections whatsoever between adjoining cuttings. (FIG. 13) This is why the glass is so important. Cutting the pedal disk 117 on any other less smooth substrate will result in incomplete separation, resulting in the coral recombining rather than separating. Or it will result in a rough cut that invites infection and coral necrosis. The glass allows a very fine clean precise cut, with complete separation and no crushing. On any other type of porous substrate, that wouldn't be possible.

It is recommended that you wear magnifying glasses to view it as you cut it. It is important not to crush or tear the coral. Cleanly slice along the natural striations of the pedal disk 117. Long thin pedal tissues can also be divided perpendicular to the striations. Often one pedal disk 117 from an average sized soft coral will yield 30-70 sliced pieces of tissue from just one adult. Use caution, since divided tissues must also stay attached to the glass for this to be successful.

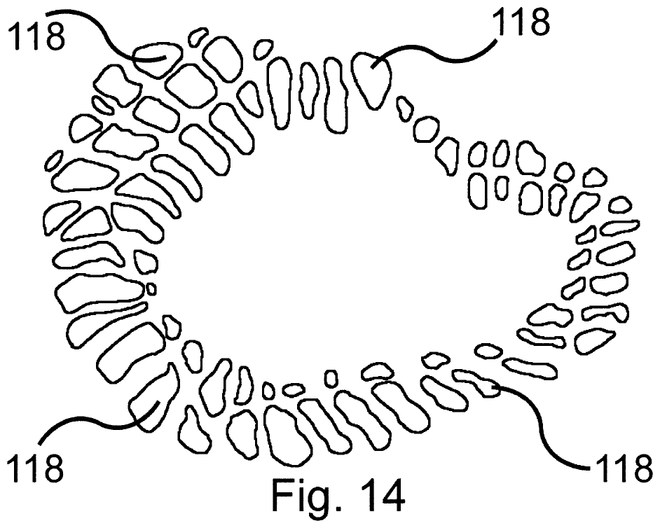

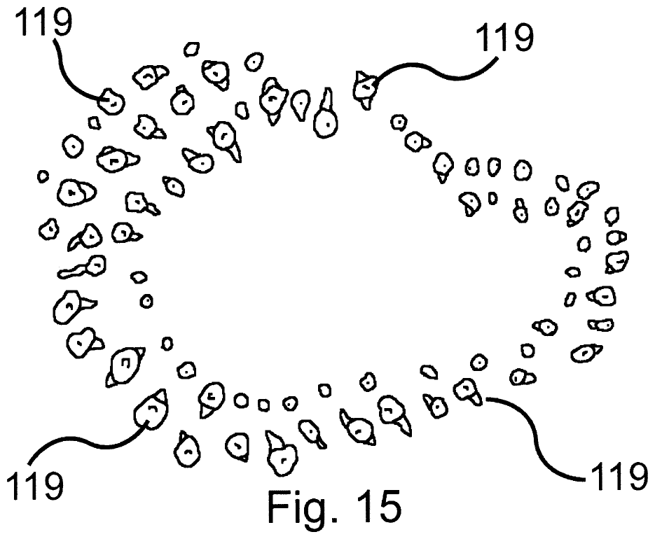

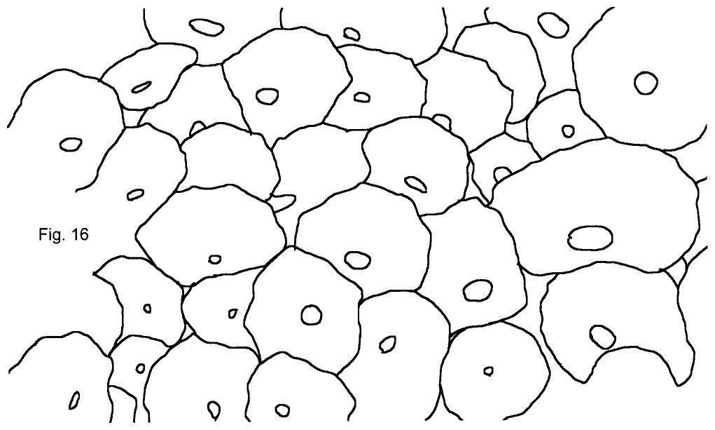

Place the glass plate 113 back in moderate flow area, in reduced light. Within a few days, each piece of tissue will begin to morph into a new coral (FIG. 14) and after a few weeks, a new stalk and oral disk will emerge out of each separate piece of soft coral cutting 108 tissue. (FIG. 15).

The glass plate 113 with the numerous juvenile corals can now be placed in the optimum position in a grow out tank.

After the tiny cuttings of pedal disks 117 have morphed into tiny adults and then grown out into full adults, it's possible to repropagate these new corals using the same method as just described. First carefully clean the glass plate 113 of all algae or other debris on both sides and around the coral pedal discs with clean razor blade. Be careful not to dislodge or undercut the pedal discs. Rinse in ambient saltwater. You should now have a clear glass plate 113 with the coral pedal disks 117 firmly attached. You can view the foot from the back side to gauge it. If not solidly attached, place back in the system and allow it to attach for a few more weeks.

In the space of a glass plate 113 the size of a sheet of A4 paper, starting out initially with 10-15 adults, you can easily create 500 to more than 1000 new juvenile corals 119 by this method on each glass plate 113, depending upon the species of corallimorphian you are growing.

This method can also be effectively used to grow numerous exact clonal copies of the coral, if limited to one original adult on the glass plate 113. This might be useful in genetic studies, where identical specimens are required.

Each juvenile will grow into an adult coral if left on the glass plate 113 and allowed to grow out. Given time, they will cover the entire glass plate 113 and crowd each other, slowing their growth. Once grown, you can repeat process ad infinitum. Or you can thin out the population to give them more room to grow, by removing some and starting other plates with the excess corals.

The key to success is coaxing the coral from its natural substrate onto a plate of glass. The glass is then an ideal backing for the precise surgical division of the pedal disk, resulting in a very large quantity of new tiny corals in very little space.

For some species, the oral disks and stalks that are cut off may be reattached to new glass plates 113 directly by following the demucus procedure and then immediately laying the cut pieces on glass plates 113 in very moderate current area, surrounded by 6 cm high×10 cm diameter PVC rings. Or if they don't attach, place the oral disk on a bed of coarse flat chunky aragonite gravel in PVC rings and allow the caps to attach to the gravel. Then repeat the process of attaching it to glass using cyanocrylate glue.

Juvenile corals can be scraped from their glass plates with their pedal disks intact, and placed either in dimple cubes or tunnel cubes, before they grow too large to fit into the dimple or tunnel.

How to Use the Cubes after Soft Coral Cuttings Become Attached

Standardizing the cube size across all methods allows them to be completely interchangeable and compatible with all methods and devices.

Whether using dimple cubes or tunnel cubes, or glass plates, at this point you have several options on what to do with them:

A—Ship the Full Intact Cube Plates.

Plates with dimple or tunnel cubes may be shipped world wide via airfreight, just as you would pack and ship wild corals. This is a very efficient way of transporting large quantities of juvenile corals since each plate contains about 117 corals. However the size of the plates, and thus number of cubes on each plate, can vary according to your system requirements.

B—Install Cubes Containing a Coral on a Natural Coral Reef.

Using any type of waterproof epoxy putty, you can embed the cubed coral dimple or tunnel cube directly on the coral reef. Placing it in a slight depression will aid the coral in attaching permanently to the reef as it grows out. Obviously consideration needs to be given regarding introducing non-native coral species into a new ecosystem.

C-Grow Out in the Lab.

It's possible to leave the tunnel plate or dimple plate as is, allowing the corals to grow out as long as desired. This is the most efficient way to grow out soft corals. At this density, one square meter of tank surface can hold up to 5200 juvenile starter corals. Grow out plates can also be further arranged as to optimize tank space. If plates are placed in rows at a 45 degree angle for grow out, one square meter of water surface can hold up to about 7500 juvenile starter corals. Soft corals grow quickly under the right conditions so they will need to be divided before long.

One effective option for spreading type soft corals such as Pacyclavularia and many of the Xenia spp. is to remove every other cube in a checkerboard pattern and replace it with a blank flat cube 101. The corals will quickly colonize the adjacent cube and double the number of corals with very little effort.

Additionally it's possible to further reduce the amount of labor and increase the production. Simply place an established spreading soft coral cube in a pattern which leaves 8 empty cubes around it allowing the coral to colonize some or all of the 8 surrounding cubes, you can quickly increase the inventory from 1 to 9 corals for each cutting. There are many cube configurations mixing blank cubes and occupied cubes, to optimize the number of soft corals grown and minimize the costs and labor.

D—Create Coral Rocks for Reef Replenishment or the Aquarium Trade.

In the grow out facility, individual dimple or tunnel cubes with corals attached can be popped out from plate, and installed in a UAR which is larger than the cube. (FIG. 17). This coral can then be grown out even further and possible encrust the rock. Done properly, the coral and rock are indiscernible from a coral on live rock as is colonized by other organisms in the saltwater system.

E—Combo Rocks (FIG. 19).

The UAR artificial rocks can have cube indentations for more than one cube, allowing mixing of different species of corals on one rock, if desired. These combo rocks (CRX) can be use to form beautiful combinations of different compatible species that don't always occur in nature, and sold to the aquarium hobby industry.

1. A solid three-dimensional cube consisting of a cementitious or similar substrate, wherein a cube consisting of an indentation (dimple) of varying sizes on top (denoted as a dimple cube-DC), to hold the soft coral cuttings (including but not limited to Pachyclavularia, Cespitularia, Xenia, Anthelia, Heteroxenia genera, and other marine or freshwater invertebrates such as sponges, molluscs, and echinoderms).

2. A method according to claim 1 wherein dimple cubes are arrayed in a polystyrene eggrate matrix or similar, containing a large number of dimple cubes denoted as a dimple cube plate (DCP).

3. A method according to claim 2 wherein soft coral cuttings are placed within the dimples of each cube, sized to be smaller than the height from the dimple bottom to the top of the dimple cube plate.

4. A method according to claim 3 wherein polyester (or similar) netting is placed on top of the dimple cube plate, netting holes being sized to contain the soft coral cuttings within each dimple 103.

5. A method according to claim 4 wherein an empty eggcrate plate of equal size to the DCP is placed on top and precisely aligned with the dimple cube plate below, with rubber bands affixed to each cross member in both directions, top and bottom, to isolate and secure the top of each dimple cube, all together denoted as a dimple cube plate assembly (DCPA).

6. A method according to claim 5 wherein the dimple cube plate assembly is placed into a saltwater system and soft corals allowed to grow and attach to the dimple cubes.

7. A solid three-dimensional cube made of a cementitious or similar substrate, wherein a cube consisting of an inner vertical tube (tunnel) of various sizes running from top to bottom, to hold the soft coral cuttings 108 (including but not limited to Sarcophyton, Palythoa, Sinularia, Zoanthus, Capnella, Protopalythoa, Cladiella, Lemnalia, Litophyton, Ricordia, Xenia, Lobophyton genera), denoted as a tunnel cube (TC).

8. A method according to claim 7 wherein tunnel cubes are arrayed in an polystyrene eggrate or similar material matrix containing a large number of tunnel cubes denoted as a tunnel cube plate (TCP).

9. A method according to claim 8 wherein fiberglass screening 109 or similar is affixed underneath the tunnel cube plate, to prevent the soft coral cuttings 108 from falling out of the tunnel as they grow and attach to the substrate, but allow oxygen and water to circulate freely within the tunnel.

10. A method according to claim 9 wherein soft coral cuttings are placed within the tunnel of each cube, falling to the bottom and resting upon the fiberglass netting 109.

11. A method according to claim 10 wherein polyester (or similar) netting 110 is placed on top of the TCP, netting holes sized in order to contain the soft coral cuttings within each tunnel.

12. A method according to claim 11 wherein an empty eggcrate plate is placed on both top and bottom and cross members precisely aligned with the middle tunnel cube plate (TCP), with rubber bands affixed to each cross member in both directions, top and bottom, to secure and separately isolate each tunnel cube, denoted as a tunnel cube plate assembly (TCPA).

13. A method according to claim 12 wherein a tunnel cube plate assembly is placed into a saltwater system and allowed to grow-out into adults.

14. Adult soft coral colonies (including but not limited to Actinodiscus, Zoanthus, Discosoma, Amplexidiscus, Rhodactis, Dendronepthya genera), with as much of their natural attached stony substrate as possible removed by tile saw without damaging the coral tissue.

15. A method according to claim 14 wherein a soft coral colony is affixed by it's remaining flat stony substrate to a glass plate surface using cyanoacrylate or similar waterproof adhesive.

16. A method according to claim 15 wherein soft coral colony over time moves off of remaining substrate naturally, and attaches firmly to glass plate only and all natural substrate is removed from the glass plate.

17. A method according to claim 16 wherein the oral disks (tentacles) and stalks of soft corals are excised, leaving only a solid pedal disk firmly attached to the glass plate, and pedal disks are then divided into numerous sections by cutting along natural striations within the disks, and further subdividing those cuttings perpendicular to the natural striation cut lines, to produce a very high volume of cuttings.

18. A method according to claim 17 wherein the glass plate is placed into a saltwater system and soft coral cuttings 108 allowed to grow-out into adults.

19. A method according to claim 17 wherein the excised oral disks (tentacles) and stalks of soft corals are placed on a bed of aragonite or similar substrate, in a low flow area of a saltwater system, surrounded by a PVC ring or similar, and allowed to reattach naturally to the substrate to begin the process again.

20. A method according to claim 18 wherein the soft corals are harvested by either removing the entire coral from the glass and individually inserting into a DCPA or TCPA, or allowing grow out and repeating steps 17-18 to begin the process again.
